(19) United States
(12) Patent Application Publication
(10) Pub. No.: US 2009/0198141 A1
(43) Pub. Date: Aug. 6, 2009

(54) SPHYGMOMANOMETER WITH DETACHABLE DATA TRANSMISSION DEVICE

(75) Inventors: Stefan Hollinger, Kronberg (DE); Dieter Wunder, Schotten (DE); Ulrich Heck, Krefeld (DE); Dirk Freund, Kelkheim (DE); Brigitte Harttmann, Niedernhausen (DE); Gerrit Roenneberg, Darmstadt (DE); Fred Schnak, Kronberg (DE)

Correspondence Address: DARBY & DARBY P.C., P.O. BOX 770, Church Street Station, New York, NY, 10008-0770, US

(73) Assignee: Kaz USA, Inc, Southborough, MA (US)

(21) Appl. No.: 11/912,723

(22) PCT Filed: Mar. 10, 2006
(86) PCT No.: PCT/EP2006/002201

§ 371 Date: Dec. 22, 2008

(30) Foreign Application Priority Data
Apr. 28, 2005 (DE) 10 2005 019 751.5

(51) Int. Cl. A61B 5/02

(52) U.S. Cl. 600/490

(57) ABSTRACT

A data transmission unit for a blood pressure meter. The data transmission unit includes an external transmission interface for the transmission of data to external data processing equipment and/or from external data processing equipment. The unit also includes an internal connection interface operable to detachably connect the data transmission unit to the blood pressure meter. As such, the data transmission unit forms an exchangeable interface converter for the blood pressure meter that converts a first data and/or transmission format fitting of the blood pressure meter into a second data and/or transmission format fitting of the external data processing device.

### CROSS-REFERENCE TO PRIOR APPLICATION

This is a U.S. National Phase application under 35 U.S.C. §371 of International Application No. PCT/EP2006/002201, filed on Mar. 10, 2006, and claims the benefit of German Patent Application No. 102005019751.5, filed on Apr. 28, 2005, both of which are incorporated herein. The International Application was published in German on Nov. 2, 2006 as WO 2006/114155 A1 under PCT Article 221(2).

### FIELD OF THE INVENTION

The present invention relates to a blood pressure meter with a measuring device for the determination of blood pressure data as well as a data transmission unit with an external transmission interface for the transmission of blood pressure data to external data processing devices and/or for the transmission of control data from external data processing devices to the measuring device.

### BACKGROUND

Blood pressure meters are increasingly confronted with the requirements of expanded data management. Such data management may serve private use and may also be part of an expanded diagnosis and/or therapy under medical supervision. Therefore, blood pressure meters include data transmission interfaces via which the measuring data stored in the blood pressure meter can be read. In practice, however, there occur problems due to the fact that the available infrastructure with regard to physicians and hospitals is not universal enough to establish itself on a broad front. There are, for example, blood pressure meters that are equipped with infrared interfaces or that can transfer data to physicians by fax modem. However, this requires that the physician in question own data processing devices that are compatible with the respective blood pressure meter. For an evaluation of the measuring data determined and/or stored in a blood pressure meter it is possible to plug a data transmission cable into a USB interface on the blood pressure meter in order to transfer the data to a PC. However, since the practice equipment of a physician is continually subject to special requirements, one may assume that, given the lack of standardization, no agreement can be achieved on a particular transmission concept.

### SUMMARY OF THE INVENTION

Therefore, the invention at hand is based on the task of creating an improved blood pressure meter as well as an improved data transfer unit for such a blood pressure meter that simplify the data transmission and improve the compatibility between the blood pressure meter and the external data processing equipment in medical practices, hospitals, etc.

The invention provides a data transmission unit for a blood pressure meter. The data transmission unit includes an external transmission interface for the transmission of data to external data processing equipment and/or from external data processing equipment. The unit also includes an internal connection interface operable to detachably connect the data transmission unit to the blood pressure meter. As such, the data transmission unit forms an exchangeable interface converter for the blood pressure meter that converts a first data and/or transmission format fitting of the blood pressure meter into a second data and/or transmission format fitting of the external data processing device.

The invention is based on the idea of separating the blood pressure meter and the data transmission unit together with its external transmission interface from each other. The blood pressure meter and data transmission unit are connectible via a connection interface so that during practical application, the blood pressure meter is not rigidly coupled to one technical data transmission form but instead can be adapted to the requirements on the part of the medical infrastructure. In accordance with an embodiment of the invention, the data transmission unit has a separate exchangeable interface converter that can be attached in separable fashion to the device body of the blood pressure meter and thus can be connected to a connection interface by means of an internal connection interface. The interface converter converts, between its internal connection interface and its external transmission interface, a first data and/or transmission format fitting the measuring device into a second data and/or transmission format fitting the respective external data processing device, and/or vice versa. The blood pressure meter may be provided with an interface adapter that can easily be exchanged so that the blood pressure meter, with regard to its data transmission interface, will be easily adaptable to the hardware used by the physician or the hospital, and in particular to country-specific data transmission modes.

Using the exchangeable interface converter, the blood pressure meter can be adapted particularly quickly to various international country standards even though the blood pressure meter can be used worldwide in the same basic configuration or, respectively, as the same basic device. The basic device is adapted to the respective required transmission standard by plugging in or, respectively, attaching the appropriate interface converter. In one embodiment, the interface converter does not only provide the geometrically fitting external transmission interface but also transforms the data format and/or the appurtenant transmission protocol used in the blood pressure meter, or more specifically, at its permanently installed connection interface into the respective required data format and transmission protocol that is preset by the interface located in the respective data processing device. It goes without saying that the data signal level can also be adapted by the interface adapter. The interface converter may be designed to work bidirectionally, i.e. it converts the first data and/or transmission format fitting of the measuring device of the blood pressure meter into the second data and/or transmission format fitting of the respective external data processing equipment, and vice versa.

In addition, the blood pressure meter can also be operated without the aforementioned interface converter, particularly if the data and transmission format provided by the permanently built-in connection interface of the blood pressure meter is already suitable for the respective external data processing device. In this case, the blood pressure meter can be connected immediately with the external data processing device via a cable in order to read the data from the blood pressure meter or, respectively, to input control data into it.

The connection interface of the blood pressure meter to which the interface converter can be connected can, in principle, be designed in various ways. In accordance with one embodiment of the invention, the connection interface can be an RS-232 port. Such an RS-232 port that is operable with personal computers, is a universal and effective interface that makes it possible to connect a multitude of forms of data transmission interfaces by means of the separate data transmission unit. In this embodiment, the internal connection interface of the interface converters is also an RS-232 port in order to be able to be connected to the corresponding firmly installed connection interface of the blood pressure meter. In this embodiment, the interface converter converts the data and transmission format specific for the RS-232 port into the data and transmission format for the external transmission interface required in each particular case. In accordance with the invention, the separate data transmission unit may have an infrared interface, a fax modem, a chip card terminal, a cable connection interface and/or a blue tooth unit as external transmission interface. Additional embodiments of the external transmission interface of the data transmission unit, such as, for example, a USB interface, a firewire port, a WLAN interface, are also possible. This makes it possible to connect the blood pressure meter to almost any external data processing equipment by simply replacing the data transmission unit in order to select data from the blood pressure meter and to process it in the data processing equipment and/or to input data from the external data processing equipment into the blood pressure meter.

Further, the interface converter can include a protocol converter that synchronizes the data transmission protocol provided at the internal connection interface with a differing data transmission protocol required at the external transmission interface. Moreover, additional data format characteristics can be converted in the required manner using a format converter in order to assure the data transmission. If necessary, a level converter can be integrated into the interface converter in order to raise and/or lower the data signal level from the internal level in the measuring device to the level required at the data processing device. If, for example, a permanently built-in RS-232 port is used in the afore-described manner at the blood pressure meter itself and a blue tooth unit is used at the data transmission unit as external transmission interface, the interface converter converts the RS-232 port-specific data format and transmission protocol to the data format and transmission protocol demanded by the blue tooth specification.

In one embodiment, the connection interface of the blood pressure meter with which the external data transmission unit is connectable can be formed by a chip card contacting unit. In this embodiment, the external data transmission unit is provided with a corresponding connection adapter that can be inserted into the chip card contacting unit. While in this embodiment the blood pressure meter is preset to the transfer medium of the chip card, it is open to any expansions by inserting the external transmission unit together with its connection adapter and data format converter. In this embodiment, the blood pressure meter can distinguish between chip card operation and interface operation, i.e. the blood pressure meter can distinguish if a chip card that can be inscribed with data has been inserted into the chip card contacting unit or if the transmission unit is being used with its connection adapter.

Advantageously, cumbersome cable connections between the device body of the blood pressure meter and the transmission unit can be dispensed with. In one embodiment of the invention, the data transmission unit may be designed as a top attachment that is connectable to the device body of the blood pressure meter via a plug-in connection.

The data transmission unit, its external transmission interface and/or the connection interface of the blood pressure meter can be designed unidirectional and in particular permit the reading of measuring data generated in the blood pressure meter.

Alternatively, the data transmission unit and/or the measuring device-internal connection interface can be designed bidirectionally. This permits, on the one hand, the aforementioned data transmission from the blood pressure meter to external data processing equipment. On the other hand, using this embodiment it is also possible to input data, in particular control data from external data processing equipment, into the blood pressure meter. For example, certain device settings such as measuring times, reminder alarms etc. can be programmed into the blood pressure meter from outside.

In accordance one embodiment, the bidirectional interface operation can be carried out by directly connecting an external data processing device, for example a computer in the form of a PC, laptop, a palmtop or the like, with the measuring device of the blood pressure meter via the connection interface instead of the aforementioned data transmission unit with external transmission interface. In this respect, the interface operation can go far beyond the pure reading or, respectively, inputting of data. If such external peripheral devices with increased calculation power and high-caliber display options are connected, the blood pressure meter merely forms an acceptance module for the measuring data. The course of the measurement can also be controlled from the external data processing device via interface commands. Likewise, the evaluation of the measuring data can take place in the external device and is not subject to any functional restrictions that might be present in a typical blood pressure meter. A considerable expansion of the performance spectrum can be achieved this way.

Alternatively or additionally, a biometric recognition device can be connected to the blood pressure meter via the interface of the blood pressure meter, in particular directly via its connection interface. The biometric recognition device can carry out a user identification, for example, using a sensor for the acceptance of a fingerprint, and then determine control data for the blood pressure meter depending on the user ID. The recognition device may then input this ID into the blood pressure meter, for example in order to adjust the properties of the measuring algorithm to the respective user.

In accordance with one embodiment of the invention, the external data transmission component can simultaneously form, together with the interface converter, a battery charger to electrically charge a battery provided for in the device body of the blood pressure meter.

However, in this case, the external exchangeable component with the interface converter can perform a double function. On the one hand, the data transmission standard of the blood pressure meter can be adapted to the requirements. At the same time, the battery of the blood pressure meter can be charged.

This embodiment is advantageous in that the battery charger is formed by an external component since this makes it possible to adapt the electrical interface of the battery charger to the respective country standards. The battery charger may have a power connection in accordance with the respective country standard and also have a level converter, for example, in order to convert the 230 V power supply voltage customary in Europe down to the charging voltage for the device-internal battery. If the device is to be adapted for the US, the battery charger containing the component will be replaced in order to reduce the required battery charging voltage to the 110 V power supply voltage common in the US.

### BRIEF DESCRIPTION OF THE DRAWINGS

These and additional characteristics will be more apparent by referring to the drawings, in which:

FIG. 1: a schematic representation of a blood pressure meter in accordance with an embodiment of the invention and a separate data transmission unit, and

FIG. 2: a schematic representation of the blood pressure meter from FIG. 1 and an external data processing device.

### DETAILED DESCRIPTION

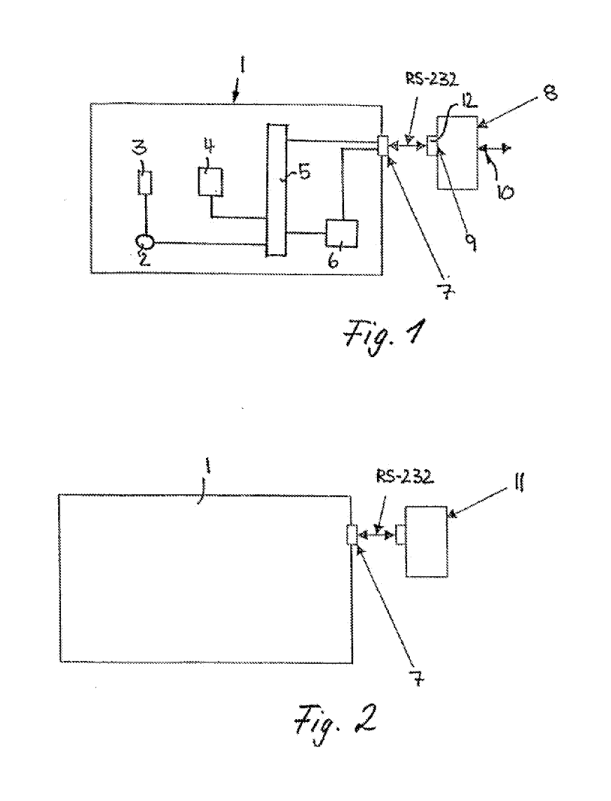

The blood pressure meter of FIG. 1, may include a pressure sleeve that can be placed around a limb and that can be filled with a fluid. In particular, the blood pressure meter can be a wrist device where the pressure sleeve can be fitted precisely around a patient's wrist.

Attached to the pressure sleeve is a device body comprising a device casing, a pump 2 for fluid filling and/or emptying of the sleeve, a power supply 3 in the form of batteries and/or storage batteries to operate the pump 2, a measuring value recorder, for example in the form of a pressure sensor 4, a control mechanism 5 to control the pump 2 and to evaluate the measuring signals of the measuring value recorders 4 as well as a storage facility 6 to store the measuring values. As shown in FIG. 1, a connection interface 7 is provided on the device body of the blood pressure meter 1 that is connected to the storage facility 6 and/or the control mechanism 5. In the embodiment as drawn, the connection interface 7 forms an RS-232 port; the connection interface 7 can advantageously be designed as a chip card contacting unit.

The transmission interface 7 can be connected to a data transmission unit 8 designed as a separate component that, on the one hand, has an internal connection interface 9 by means of which it can be connected to the RS-232 interface 7 of the blood pressure meter. On the other hand, the data transmission unit 8 comprises an external data transmission interface 10 that, in principle, can have varying formats. The external data transmission interface 10 can be, for example, an infrared transmission interface, a fax modem, a blue tooth unit, a chip card terminal and/or a connecting cable terminal.

Advantageously, the data transmission unit 8 has an interface converter that functions between the internal connection interface 9 and the external transmission interface 10. The interface converter transforms the data and transmission format required at the internal connection interface which is conditioned by the connection to the RS-232 interface 7 of the blood pressure meter into the proper data and transmission format that is required at the external data transmission interface 10 and that is conditioned by the external data processing equipment that is to be connected thereto. To this end, the interface converter not only adapts the voltage level of the individual data signals to the requirements but also transforms the data format and the transmission protocol, for example from the RS232 format to the format prescribed by the blue tooth specifications.

Advantageously, the data transmission unit 8 can be connected to the device body of the blood pressure meter by means of a plug-in connection. In particular, the internal connection interface 9 of the data transmission unit 8 can be a chip card adapter that can be plugged into the connection interface 7 of the device body of the blood pressure meter 1 designed as a chip card contacting unit. This makes it possible to replace the data transmission unit 8 in a simple manner.

As shown in FIG. 2, the design of the connection interface 7 of the device body of the blood pressure meter 1 in the form of a chip card contacting unit also provides the advantage that an external data processing device, for example in the form of a laptop or of a PC, can be connected directly to the connection interface 7 of the device body of the blood pressure meter. Going beyond pure data transmission, this allows the execution of the evaluation of the measuring data determined by the blood pressure meter and the control of the blood pressure meter on the external data processing device 11 in the manner described above.

1. A blood pressure meter comprising:
a measuring device obtaining blood pressure data;
a device body connection interface;
a data transmission unit that is separable from the device body including:
an internal connection interface connectable to the device body connection interface,
an external transmission interface for at least one of the transmission of the blood pressure data to external data processing devices and the transmission of control data to the measuring device, and
an interface converter that converts a first data and/or transmission format fitting corresponding to the measuring device into a second data and/or transmission format fitting corresponding to the external data processing device.

2. The blood pressure meter recited in claim 1, wherein the device body connection interface comprises a RS-232 port.

3. The blood pressure meter recited in claim 1, wherein the device body connection interface comprises a chip card contacting unit.

4. The blood pressure meter recited in claim 1, wherein the interface converter comprises a protocol converter operable to synchronize a data transmission protocol of the internal connection interface with differing data transmission protocol of the external transmission interface.

5. The blood pressure meter recited in claim 1, wherein the interface converter comprises a level converter operable to adapt a data signal level of the internal connection interface to a differing data signal level of the external transmission interface.

6. The blood pressure meter recited in claim 1, wherein the external transmission interface comprises at least one of an infrared interface, a fax modem, a chip card terminal, a cable connection interface, a blue tooth unit, a USB interface, a firewire port and a WLAN interface.

7. The blood pressure meter recited in claim 1, wherein the data transmission unit comprises a battery charger operable to electrically charge a battery included in the device body of the blood pressure meter.

8. The blood pressure meter recited in claim 1, wherein the internal connection interface and the external connection interface are different from each other.

9. The blood pressure meter recited in claim 1, wherein the data transmission unit comprises a top attachment operable to be connected to the device body of the blood pressure meter via a plug-in connection.

10. The blood pressure meter recited in claim 1, wherein the data transmission unit is bidirectional.

11. The blood pressure meter recited in claim 1 designed as a wrist device.

12. A data transmission unit for a blood pressure meter comprising:
an external transmission interface for the transmission of data at least one of to external data processing equipment and from external data processing equipment, and
an internal connection interface operable to detachably connect the data transmission unit to the blood pressure meter,
wherein the data transmission unit forms an exchangeable interface converter for the blood pressure meter that converts, between its internal connection interface and its external transmission interface, a first data and/or transmission format fitting of the blood pressure meter into a second data and/or transmission format fitting of the external data processing device.

13. The data transmission unit recited in claim 12, wherein the internal connection interface comprises an RS-232 port.

14. The data transmission unit recited in claim 12, wherein the internal connection interface comprises a chip card adapter.

15. The data transmission unit recited in claim 12, wherein the external transmission interface comprises at least one of an infrared interface, a fax modem, a chip card terminal, a cable connection interface, a USB interface, a firewire port, a WLAN interface and a blue tooth unit.
